(19) United States
(12) Patent Application Publication
(10) Pub. No.: US 2010/0087662 A1
(43) Pub. Date: Apr. 8, 2010

(54) CHIRAL SALEN CATALYSTS AND METHODS FOR THE PREPARATION OF CHIRAL COMPOUNDS FROM RACEMIC EPOXIDES BY USING THEM

(76) Inventor: Ho Seong Lee, Daejeon (KR)

Correspondence Address: CLARK & BRODY, 1090 VERMONT AVENUE, NW, SUITE 250, WASHINGTON, DC, 20005, US

(21) Appl. No.: 12/522,058

(22) PCT Filed: May 23, 2008
(86) PCT No.: PCT/KR08/02895

§ 371 Date: Jul. 2, 2009

(30) Foreign Application Priority Data
Jun. 13, 2007 (KR) 10-2007-0057857

(51) Int. Cl. C07D 327/10; C07F 17/00; C07D 301/27; C07C 31/20

(52) U.S. Cl. 549/3; 556/33; 549/514; 568/852

(57) ABSTRACT

The present invention relates to new chiral salen catalysts and the preparation method of chiral compounds from racemic epoxides using the same. More specifically, it relates to new chiral salen catalysts that have high catalytic activity due to new molecular structures and have no or little racemization of the generated target chiral compounds even after the reaction is completed and can be also reused without catalyst regeneration treatment, and its economical preparation method to mass manufacture chiral compounds of high optical purity, which can be used as raw materials for chiral food additives, chiral drugs, or chiral crop protection agents, etc., using the new chiral salen catalysts.

### TECHNICAL FIELD

The present invention relates to new chiral salen catalysts and the preparation method of chiral compounds from racemic epoxides using the same. More specifically, it relates to new chiral salen catalysts that have high catalytic activity due to new molecular structures and have no or little racemization of the generated target chiral compounds even after the reaction is completed and can be also reused without catalyst regeneration treatment, and its economical preparation method to mass manufacture chiral compounds of high optical purity, which can be used as raw materials for chiral food additives, chiral drugs, or chiral crop protection agents, etc., using the new chiral salen catalysts.

### BACKGROUND ART

Chiral compounds such as chiral epoxides or chiral 1,2-diol are main ingredient materials used to synthesize drugs, crop protection agents, and food additives having optical activity [U.S. Pat. No. 5,071,868; Tetrahedron Lett., Vol. 28(16), 1783, 1987; J. Org. Chem., Vol. 64, 8741, 1999]. These chiral epoxides or chiral 1,2-diol having high optical activity are industrially very useful but had limited usages, because the preparation methods have been so far difficult or incomplete and it was hard to mass manufacture them industrially with less cost and they had no high optical purity, which is most important for product quality. As for the optical purity for a chiral intermediate needed as a raw material for drugs, the products with 99.5% optical purity or higher are considered as suitable or accepted due to its technical difficulty in preparation and limit in measurement technology of high optical purity.

The method to prepare chiral epichlorohydrin as one of chiral epoxides, with the raw material of chiral sulfonyloxyhaloalcohol derivatives obtained from mannitol derivatives [U.S. Pat. No. 4,408,063; J. Org. Chem. Vol. 43, 4876, 1978] and the preparation method using chiral 3-chloro-1,2-propanediol [Synlett, No. 12, 1927, 1999] are multistage reactions and do not have enough productivity and economical efficiency to be used industrially. Another preparation method using microorganism [European Patent No. 431,970; Japanese Laid-Open Patent Publication No. 90-257,895, No. 94-211,822] has also low productivity compared to its reaction scale and it is not economically effective because of the multistage process with more than two steps.

As other methods to prepare chiral epoxides, there are some disclosed methods on how to use chiral catalysts having stereoselectivity. With inexpensive racemic compounds mixed with optical isomers on the ratio of 50:50, these methods obtain the desired isomers by making only one isomer react and then separating the reacted isomers and not-reacted ones using their differences in physical properties through the resolution method of reaction kinetics under the existence of chiral catalysts.

Among the disclosed methods to prepare chiral epoxides by the resolution method of reaction kinetics using chiral catalysts, the methods in Bulletin of the Chemical Society of Japan, Vol. 48(6), 1897, 1975; Tetrahedron, Vol. 36, 3391, 1980; Bulletin of the Chemical Society of Japan, Vol. 52(9), 2614, 1979; Chemistry Letters, 645, 1976 are all the methods that use chiral salen catalysts and cannot be used industrially due to very low optical purity of the obtained chiral epoxides.

On the other hand, Eric N. Jacobsen et. al. disclosed the method to separate not-reacted chiral epoxides and ring-opened chiral 1,2-diol respectively or selectively, by selective hydrolysis of one optical isomer out of R-type or S-type after racemic epoxides are added with water under the existence of chiral salen catalysts [U.S. Pat. No. 5,637,739, No. 5,663,393, No. 5,665,890, No. 5,929,232, No. 6,262,278, No. 6,448,414, No. 6,800,766, International Patent Publication No. WO 91/14694, No. WO 00/09463, Korean Patent No. 473,698]. It is described that the method, compared to the existing method using chiral catalysts, has higher yield and relatively greater optical purity.

On Page 86˜87 of International Patent Publication No. WO 00/09463, however, it is described that after hydrolysis of racemic epoxides using chiral salen catalysts, as described in the said reference, the side reaction of the products generated by hydrolysis causes racemization of chiral epoixdes, which happens more and more as time passes. Thus, the method also has a limit as a preparation method of chiral epoxide compounds with high optical purity. In the said method, racemic epoxides are added with chiral salen catalysts and then added with water, then after selective hydrolysis of one optical isomer out of R-type or S-type, not-reacted (unhydrolyzed) chiral epoxides are colleted through the purification process. During the purification process, racemization is caused due to side reaction of the hydrolysis product (chiral 1,2-diol), which is a side effect due to instability of the used chiral salen catalysts, being a fatal drawback to mass production of chiral epoxides. In case of mass manufacturing, it takes longer time to purify in proportional to the volume of reactants, resulting in lower optical purity of target chiral epoxides compared to the product quantity. Thus, there is a limit to apply the disclosed chiral salen catalysts for mass manufacturing of chiral epoxides having high optical purity, which is suitable for medical or food additive use. Also, it was claimed that racemization could be reduced by using tetrahydrofuran as a solvent [U.S. Pat. Nos. 6,448,414 and 6,800,766], but using tetrahydrofuran as a solvent makes reaction very slow and takes additional purification time to eliminate tetrahydrofuran after reaction is finished, which is bad for mass manufacturing.

In case of the existing chiral salen catalyst by Eric N. Jacobsen, when it is reused, its catalytic activity deteriorates due to instability of the catalyst, so the catalyst has to be collected every time for additional regeneration process. Also, even after the regeneration process, its activity is remarkably low compared to the optical purity of chiral epoxides prepared by new ones, so the number of reuse is also limited. Such disadvantages are critical reasons for raising the production cost of chiral compounds to be used industrially, and furthermore may prohibit them from being applied industrially.

Therefore, chiral compounds such as chiral epoxides or chiral 1,2-diol are important materials to prepare various drugs, food additives, crop protection agents, etc., but have many limits in their preparation methods, so there is a desperate need for the development of more effective preparation methods that are more industrially useful than the existing technologies in order to prepare chiral compounds having high optical purity.

Also, the disclosed chiral salen catalysts including that of Eric N. Jacobsen have been studied focusing on chiral salen ligand and there have been no references on the example where anionic ligand was added with new functions to increase catalytic activity or stereoselectivity.

### DISCLOSURE OF INVENTION

### Technical Subject

An object of the present invention is to provide new chiral salen catalysts that have high catalytic activity due to new molecular structures and have no or little racemization of the generated target chiral compounds even after the reaction is completed and can be also reused without catalyst regeneration treatment.

Also, the object of the present invention is to provide the preparation method of new chiral salen catalysts that have high catalytic activity due to new molecular structures and have no or little racemization of the generated target chiral compounds even after the reaction is completed and can be also reused without catalyst regeneration treatment.

Also, another object of the present invention is to provide the preparation method to economically mass manufacture chiral compounds such as chiral epoxides or chiral 1,2-diol, which have high optical purity and high yield by stereoselective hydrolysis of racemic epoxides using new chiral salen catalysts.

### Technical Solution

The present invention provides new chiral salen catalysts presented as Chemical Formula 1 as below, which are the catalysts for reaction to prepare chiral compounds such as chiral epoxides or chiral 1,2-diol by stereoselective hydrolysis of racemic epoxides.

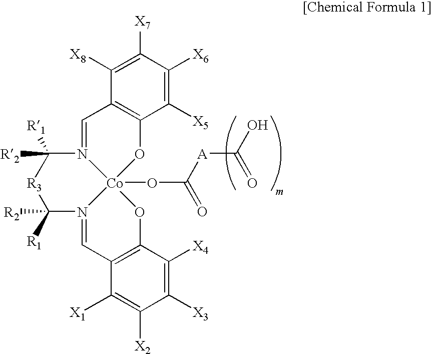

### [In Chemical Formula 1:

R₁, R₂, R′₁, R′₂, X₁, X₂, X₃, X₄, X₅, X₆, X₇ and X₈ are independently hydrogen atom, linear or branched saturated or unsaturated (C1-C7) alkyl group, (C1-C7) alkoxy group, halogen atom, hydroxy group, amino group, thiol group, nitro group, aminocarbonyl, (C3-C7) cycloalkyl, (C1-C7)alkoxy(C1-C7)alkyl, (C1-C7) alkylcarbonyl, (C1-C7) alkoxycarbonyl, (C3-C7)cycloalkyl(C1-C7)alkoxy, mono or di(C1-C7)alkylamino, (C1-C7) alkylcarbonylamino, t-butoxycarbonylamino, phthalimido, carboxylic group, aldehyde group, (C1-C7) alkylthio, (C1-C7) alkylsulfonyl group, tri(C1-C7) alkylsilyl group, tri(C6-C10)arylsilyl group, mono or di(C1-C7) alkylaminocarbonyl, —(CH₂)ₖ—R₄, or (C2-C10)alkylene to form a ring by combining with adjacent substituents;
R₃ is a direct bond, (C1-C5)alkylene, —NH—, —O—, or —S—;
R₄ is a 3 to 5-membered saturated or unsaturated heterocycle including N, O, or S, (C3-C12) cycloalkyl, or phenyl;
A is (C1-C12)alkylene, which can be more substituted with linear or branched saturated or unsaturated (C1-C7) alkyl group, (C1-C7) alkoxy group, halogen atom, hydroxy group, amino group, thiol group, (C1-C7) alkylcarbonylamino, t-butoxycarbonylamino, phthalimido, —O₂CY₃, or —O₃SY₃, or can form cycles by being connected with (C2-C10)alkylene, —OSO₂—, —OSO₃—, or —OCO₂—;
Y₃ is a linear or branched saturated or unsaturated (C1-C7)alkyl group or phenyl, which can be more substituted with linear or branched saturated or unsaturated (C1-C7)alkyl group, halogen, or nitro;
k is an integer of 0 to 8;
m is an integer of 1 to 3.]

For example, R₁, R₂, R′₁, R′₂, X₁, X₂, X₃, X₄, X₅, X₆, X₇ and X₈ can be independently hydrogen atom, methyl, ethyl, n-propyl, i-propyl, n-butyl, i-butyl, t-butyl, pentyl, hexyl, ethenyl, propa-1,2-dienyl, ethinyl, methoxy, ethoxy, propoxy, butoxy, pentoxy, hectoxy, bromo, chloro, fluoro, hydroxy, amino, thiol, nitro, aminocarbonyl, cyclopropyl, cyclobutyl, cyclohexyl, methoxymethyl, methoxyethyl, butoxyethyl, butoxypropyl, methylcarbonyl, ethylcarbonyl, butylcarbonyl, methoxycarbonyl, ethoxycarbonyl, butoxycarbonyl, cyclopropylmethoxy, cyclobutylmethoxy, cyclohexylmethoxy, methylamino, dimethylamino, methylethylamino, acetylamino, t-butoxycarbonylamino, phthalimido, carboxylic, aldehyde group, methylthio, ethylthio, methylsulfonyl, ethylsulfonyl, t-butylsulfonyl, trimethylsilyl, dimethylethylsilyl, triphenylsilyl, methylaminocarbonyl, ethylaminocarbonyl, phenyl, or benzyl; R₁, R₂, R′₁, R′₂, X₁, X₂, X₃, X₄, X₅, X₆, X₇ and X₈ can independently form 5-membered, 6-membered or 7-membered rings by being connected with adjacent substituents.

The new chiral salen catalyst of the present invention is presented more preferably as Chemical Formula 2 below.

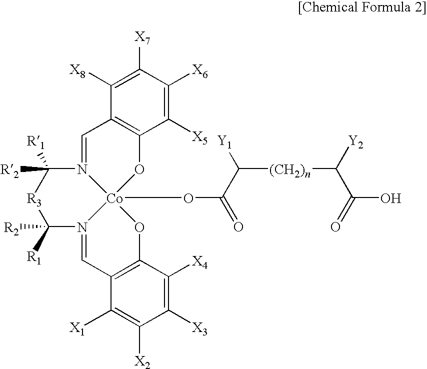

### [In Chemical Formula 2:

R₁, R₂, R₃, R′₁, R′₂, X₁, X₂, X₃, X₄, X₅, X₆, X₇ and X₈ are identical as those of Chemical Formula 1;
Y₁ and Y₂ are independently hydrogen atom, linear or branched saturated or unsaturated (C1-C7) alkyl group, (C1-C7)alkoxy group, halogen atom, hydroxy group, amino group, thiol group, (C1-C7) alkylcarbonylamino, t-butoxycarbonylamino, phthalimido, —O₂CY₃, or —O₃SY₃, or Y₁ and Y₂ can form cycles by being connected with (C2-C10)alkylene, —OSO₂—, —OSO₃—, or —OCO₂—;
Y₃ is a linear or branched saturated or unsaturated (C1-C7) alkyl group or phenyl, which can be substituted more with linear or branched saturated or unsaturated (C1-C7) alkyl group, halogen, or nitro;
n is an integer of 0 to 10.]

In Chemical Formula 1 and 2, it is preferable that X₁, X₂, X₃, X₄, X₅, X₆, X₇ and X₈ are independently selected from the group consisting of hydrogen atom, linear or branched saturated or unsaturated (C1-C7) alkyl group, and (C1-C7)alkoxy group, and it is more preferable that X₁, X₂, X₃, X₄, X₅, X₆, X₇ and X₈ are independently hydrogen atoms or t-butyl groups.

In Chemical Formula 1 and 2, R₁ and R′₁ can be either identical or different, but it is preferable to be identical. R₂ and R′₂ can be also either identical or different, but it is preferable to be identical, too. When R₁ and R′₁ are identical and R₂ and R′₂ also identical, the chiral center then has R₁, configuration or SS configuration. The preferable examples of R₁, R′₁, R₂, and R′₂ are the case when R₁ and R′₁ are combined to (C2-C8)alkylene to form a ring and R₂ and R′₂ are hydrogen atoms or another case when R₂ and R′₂ are combined to (C2-C8)alkylene to form a ring and R₁ and R′₁ are hydrogen atoms.

In Chemical Formula 2, it is preferable that Y₁ and Y₂ are independently hydrogen atom, (C1-C7)alkoxy group, halogen atom, hydroxy group, —O₂CY₃ or —O₃SY₃, or Y₁ and Y₂ can form a ring by being connected with —OSO₂—, —OSO₃—, or —OCO₂—; Y₃ is a linear or branched saturated or unsaturated (C1-C7) alkyl group, phenyl, nitrophenyl, which can be substituted more with linear or branched saturated or unsaturated (C1-C7) alkyl group or halogen. It is more preferable that Y₁ and Y₂ are independently C1-C7)alkoxy group, —O₂CY₃, or —O₃SY₃, and Y₃ is a linear or branched saturated or unsaturated (C1-C7) alkyl group or phenyl, which can be substituted more with linear or branched saturated or unsaturated (C1-C7) alkyl group or halogen.

Also, the present invention comprises the preparation method of chiral compounds that use the chiral salen catalyst presented as Chemical Formula 1 or 2 above as its catalyst, regarding the method to prepare chiral compounds of non-reacted chiral epoxides and hydrolyzed chiral 1,2-diol by stereoselective hydrolysis of racemic epoxides.

The present invention is described in more detail.

The present invention relates to the new chiral salen catalysts presented as Chemical Formula 1 or 2 that have high catalytic activity due to new molecular structures and have no or little racemization of the generated target chiral compounds even after the reaction is completed and can be also reused without catalyst regeneration treatment, and the preparation method of chiral compounds such as chiral epoxides or chiral 1,2-diol, which have high optical purity and high yield by stereoselective hydrolysis of racemic epoxides in the presence of the said new chiral salen catalysts.

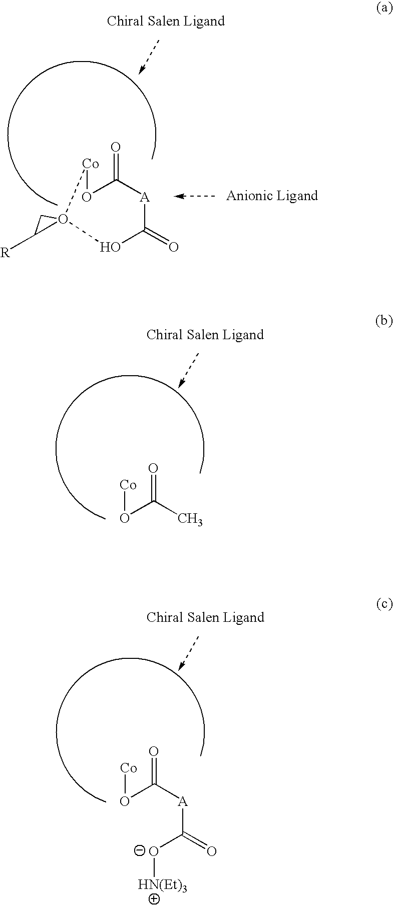

The new chiral salen catalyst (a) of the present invention has a carboxylic acid group at the end of anionic ligand, so racemic epoxides get near the chiral salen catalyst (a) of the present invention as above and they go toward the carboxylic acid group, accelerating ring opening reaction of racemic epoxides and increasing stereoselectivity.

That is, the new chiral salen catalyst (a) of the present invention contains the carboxylic acid group at the end of anionic ligand and shows improved reaction rate and better stereoselectivity than the existing chiral salen catalyst (b) containing acetic acid group by Eric N. Jacobsen, which has no carboxylic acid groups at the end. Also, it shows remarkably improved reaction rate and better stereoselectivity than the catalyst (c) which has the same structure but no carboxylic acid group. It is found that these results are shown by the act of acid in the carboxylic acid group, where exists at the end of anionic ligand of the new chiral salen catalyst (a) of the present invention.

The said chiral salen catalyst presented as Chemical Formula 1 can be prepared, as shown in Scheme 1 below, by reacting the compound presented as Chemical Formula 6 with cobalt acetate in suitable organic solvents, followed by filtering it to obtain the compound presented as Chemical Formula 3, and then reacting the resulted solid compound with the compound presented as Chemical Formula 4 in suitable organic solvents.

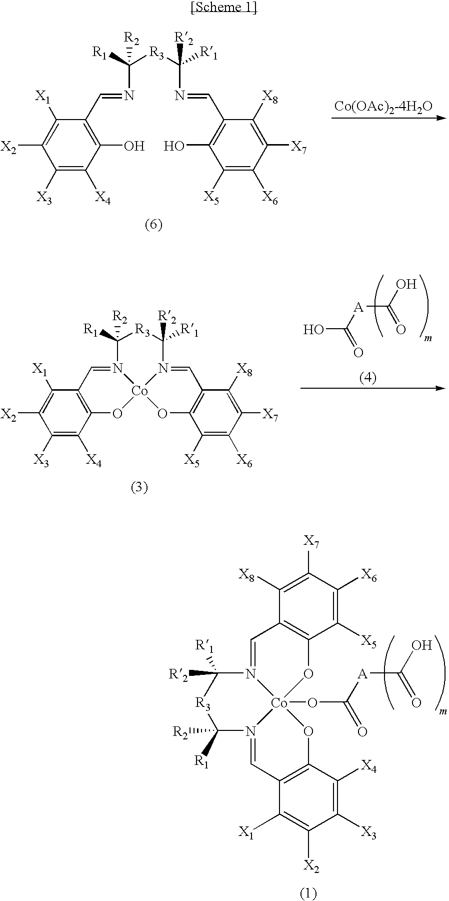

[In Scheme 1: R₁, R₂, R₃, R′₁, R′₂, X₁, X₂, X₃, X₄, X₅, X₆, X₇, X₈, A and m are identical as those of Chemical Formula 1.]

The compound presented as Chemical Formula 6 used for the preparation of chiral salen in Scheme 1 can be purchased or prepared by applying the disclosed methods [J. Org. Chem., Vol. 59, 1939, 1994]. Also, the compound presented as Chemical Formula 4 can be purchased or prepared by the usual chemical synthesizing methods.

The new chiral salen catalysts of the present invention can be used as they are or fixed on specific stationary phases such as zeolite, silica gel, resin, etc. Fixation can be achieved by physical adsorption or chemical bonding using linkers or spacers.

Since the present invention includes the preparation method of chiral compounds using the new chiral salen catalysts presented as Chemical Formula 1 or 2 above, the Scheme 2 below shows the preparation method of chiral compounds such as chiral epoxides or chiral 1,2-diol by stereoselective hydrolysis of racemic epoxides using the new chiral salen catalysts of the present invention.

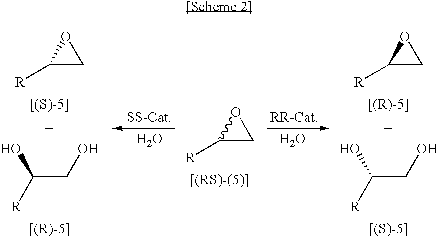

### [In Scheme 2:

R is a linear or branched saturated or unsaturated (C1-C7) alkyl group, (C3-C7)cycloalkyl group, (C1-C7)alkoxy group, phenyl group, carboxylic group, aldehyde group, (C3-C7)cycloalkyl, (C1-C7)alkoxy(C1-C7)alkyl, (C1-C7)alkylcarbonyl, (C1-C7) alkoxycarbonyl, (C3-C7)cycloalkyl(C1-C7)alkoxy, (C1-C7)alkylsulfonyl group, or —(CH₂)ₖ—R₅; the said alkyl, cycloalkyl, alkoxy, or phenyl can be substituted more with halogen;
R₅ is a linear or branched saturated or unsaturated (C1-C7) alkyl group, (C1-C7)alkoxy group, phenyl group, (C3-C7)cycloalkyl group, 3 to 5-membered saturated or unsaturated heterocycle including N, O, or S, halogen atom, hydroxy group, amino group, thiol group, nitro group, aminocarbonyl, mono or di(C1-C7)alkylaminocarbonyl, (C3-C7)cycloalkyl, (C1-C7)alkoxy(C1-C7)alkyl, (C1-C7)alkylcarbonyl, (C1-C7)alkoxycarbonyl, (C3-C7)cycloalkyl(C1-C7)alkoxy, (C6-C10)aryloxy, benzyloxy, (C1-C7)alkylcarbonyloxy, mono or di(C1-C7)alkylamino, (C1-C7)alkylcarbonylamino, t-butoxycarbonylamino, phthalimido, carboxylic group, aldehyde group, (C1-C7)alkylthio, (C1-C7)alkylsulfonyl group, tri(C1-C7)alkylsilyl group, or tri(C6-C10)arylsilyl group;
k is an integer of 0 to 8; RR-Cat. and SS-Cat. are chiral salen catalysts having RR configuration and SS configuration respectively among the chiral salen catalysts presented as Chemical Formula 1.]

The stereoselective hydrolysis reaction of Scheme 2 is performed as follows. Racemic epoxides presented as Chemical Formula (RS)-5 react with water under the new chiral salen catalysts of the present invention. Only one out of (R)-epoxides and (S)-epoxides is selectively hydrolyzed and then non-reacted epoxides and hydrolyzed 1,2-diol are separated in sequence. More detailed description is as follows.

First, to the racemic epoxide presented as Chemical Formula (RS)-5, 0.001 mole % or more (preferably 0.1˜2 mole %) of the new chiral salen catalyst of the present invention is added and then 0.3˜0.8 equivalent of water is slowly added with at −5˜40° C. (preferably 0˜25° C.). After the reaction is finished, using fractional distillation under reduced pressure or a thin film evaporator, non-reacted chiral epoxides and hydrolyzed chiral 1,2-diol are separated at −10˜70° C. (preferably 0˜30° C.). With no catalyst regeneration processes followed, new racemic epoxides are put into the reactor, wherein water is slowly added. The same process keeps repeated to synthesize more chiral epoxides or chiral 1,2-diol.

The stereoselective hydrolysis in the present invention may be changed according to the nomenclature, but when chiral salen catalysts having RR configuration are used among new chiral salen catalysts of the present invention, (R)-epoxides and (S)-1,2-diol are generated. On the other hand, when those having SS configuration are used, (S)-epoxides and (R)-1,2-diol are generated.

### BRIEF DESCRIPTION OF DRAWINGS

FIG. 1 is a graph showing the difference in the reaction rate depending on the location of the carboxylic acid group of anionic ligand among the chiral salen catalysts of the present invention.

FIG. 2 is a graph showing the difference in the reaction rate depending on the structure of each catalyst to see the change depending on stereo structure of anionic ligand among the chiral salen catalysts of the present invention.

FIG. 3 is a graph showing the difference in the reaction rate depending on the structure of each catalyst to see the change depending on the length of anionic ligand among the chiral salen catalysts of the present invention.

FIG. 4 is a graph comparing the effect of the present invention's representative chiral salen catalyst [(1-RR)-(Dibenzoyl-LTA)] and Eric N. Jacobsen's representative chiral salen catalyst (Comparative Catalyst 1) containing acetic acid groups (OAc) on racemization of chiral epoxides generated after the reaction.

FIG. 5 is a graph showing the change of stereoselectivity depending on the reaction rate and the reuse number of the present invention's representative chiral salen catalyst [(1-RR)-(Dibenzoyl-LTA)].

FIG. 6 is a graph showing the change of stereoselectivity depending on reuse of the existing chiral salen catalyst (Comparative Catalyst 1) containing acetic acid groups (OAc).

FIG. 7 is a graph showing the difference in the reaction rate depending on the structure of each catalyst to see the role of acid in the carboxylic acid group of anionic ligand among the chiral salen catalysts of the present invention.

### BEST MODE

Hereinafter, the present invention is described in more detail based on the preparation examples and the examples. But, these examples are not intended to limit the scope of the present invention.

### Preparation Example 1

### Preparation of Catalyst [(1-RR)-(Dibenzoyl-LTA)]

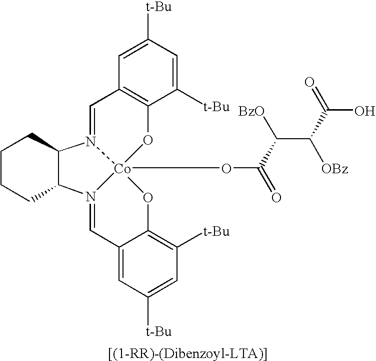

27.8 g of (R,R)—N,N′-bis(3,5-di-t-butylsalicylidene)-1,2-cyclohexanediamine and 16.4 g of cobalt(II)acetate-4H₂O were mixed in 500 ml of ethanol and stirred under reflux for 5 hours. They were filtered at room temperature and cleaned by small amount of ethanol. The obtained solid, 17.9 g of dibenzoyl-L-tartaric acid, 120 ml of acetone, and 600 ml of dichloromethane were all mixed and stirred for 5 hours with injection of air at room temperature. After the solvent was eliminated under reduced pressure, the title compound was obtained quantitatively.

mp 135° C. (dec.)

IR (KBr) 3446, 2954, 2867, 1729, 1638, 1605, 1528, 1438, 1362, 1261, 1202, 1176, 713 cm⁻¹

### Preparation Example 2

### Preparation of Catalyst [(1-SS)-(Dibenzoyl-DTA)]

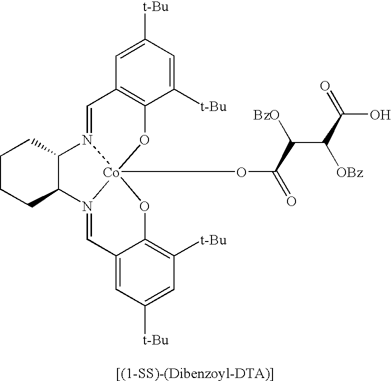

The same method as Preparation Example 1 was conducted except using (S,S)—N,N′-bis(3,5-di-t-butylsalicylidene)-1,2-cyclohexanediamine and dibenzoyl-D-tartaric acid instead of (R,R)—N,N′-bis(3,5-di-t-butylsalicylidene)-1,2-cyclohexanediamine and dibenzoyl-L-tartaric acid. Then the title compound was obtained quantitatively.

mp 135° C. (dec.)

IR (KBr) 3446, 2954, 2867, 1729, 1638, 1605, 1528, 1438, 1362, 1261, 1202, 1176, 713 cm⁻¹

### Preparation Example 3

### Preparation of Catalyst [(1′-RR)-(Dibenzoyl-LTA)]

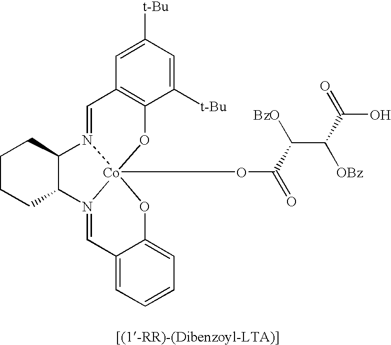

The same method as Preparation Example 1 was conducted except using 22.1 g of (R)—N-(3,5-di-t-butylsalicylidene)-(R)—N′-(salicylidene)-1,2-cyclohexanediamine instead of (R,R)—N,N′-bis(3,5-di-t-butylsalicylidene)-1,2-cyclohexanediamine. Then the title compound was obtained quantitatively.

IR (KBr) 3446, 2954, 2867, 1729, 1638, 1605, 1528, 1438, 1362, 1261, 1202, 1176, 713 cm⁻¹

### Preparation Example 4

### Preparation of Catalyst [(1′-SS)-(Dibenzoyl-LTA)]

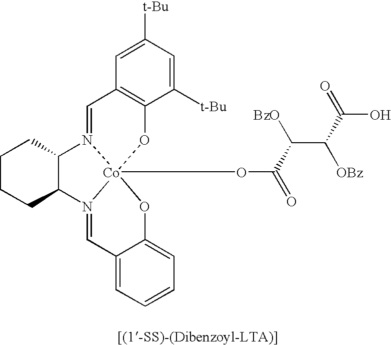

The same method as Preparation Example 1 was conducted except using 22.1 g of (S)—N-(3,5-di-t-butylsalicylidene)-(S)—N′-(salicylidene)-1,2-cyclohexanediamine instead of (R,R)—N,N′-bis(3,5-di-t-butylsalicylidene)-1,2-cyclohexanediamine. Then the title compound was obtained quantitatively.

IR (KBr) 3446, 2954, 2867, 1729, 1638, 1605, 1528, 1438, 1362, 1261, 1202, 1176, 713 cm⁻¹

### Preparation Example 5

### Preparation of Catalyst [(1-RR)-(Diacetyl-LTA)]

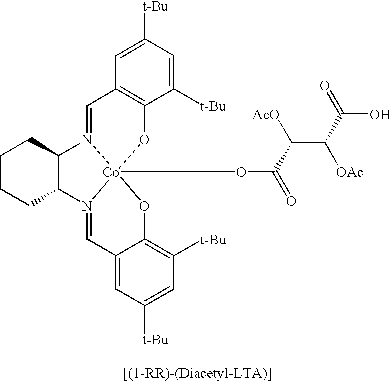

27.8 g of (R,R)—N,N′-bis(3,5-di-t-butylsalicylidene)-1,2-cyclohexanediamine and 16.4 g of cobalt(II)acetate-4H₂O were mixed in 500 ml of ethanol and stirred under reflux for 5 hours. They were filtered at room temperature and cleaned by small amount of ethanol. The obtained solid, 11.7 g of diacetyl-L-tartaric acid, 60 ml of acetone, and 600 ml of dichloromethane were all mixed and stirred for 5 hours with injection of air at room temperature. After the solvent was eliminated under reduced pressure, the title compound was obtained quantitatively.

mp 125° C. (dec.)

IR (KBr) 3536, 2956, 2866, 1750, 1635, 1611, 1528, 1439, 1367, 1251, 1220, 1173, 1066, 931 cm⁻¹

### Preparation Example 6

### Preparation of Catalyst [(1-RR)-(Diacetyl-DTA)]

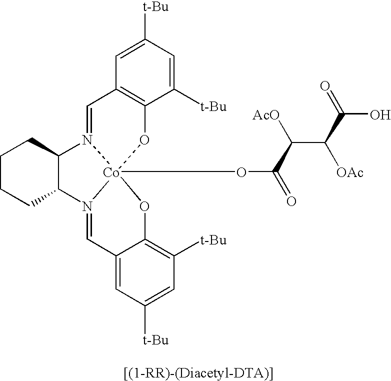

The same method as Preparation Example 5 was conducted except using diacetyl-D-tartaric acid instead of diacetyl-L-tartaric acid. Then the title compound was obtained quantitatively.

mp 126° C. (dec.)

IR (KBr) 3535, 2957, 2866, 1754, 1630, 1611, 1528, 1440, 1363, 1251, 1219, 1173, 1048, 932 cm⁻¹

### Preparation Example 7

### Preparation of Catalyst [(1-RR)-(LTA)]

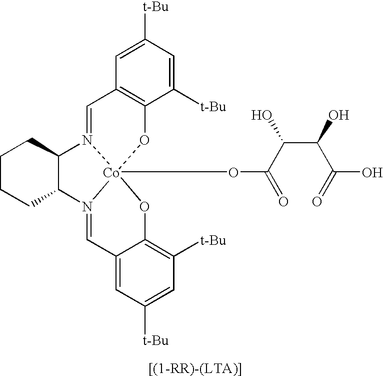

27.8 g of (R,R)—N,N′-bis(3,5-di-t-butylsalicylidene)-1,2-cyclohexanediamine and 16.4 g of cobalt(II)acetate-4H₂O were mixed in 500 ml of ethanol and stirred under reflux for 5 hours. They were filtered at room temperature and cleaned by small amount of ethanol. The obtained solid, 7.5 g of L-tartaric acid, 600 ml of acetone, and 600 ml of dichloromethane are mixed and stirred for 5 hours with injection of air at room temperature. After the solvent was eliminated under reduced pressure, the title compound was obtained quantitatively.

mp>150° C. (dec.)

### Preparation Example 8

### Preparation of Catalyst [(1-RR)-(Sulfinylated-LTA)]

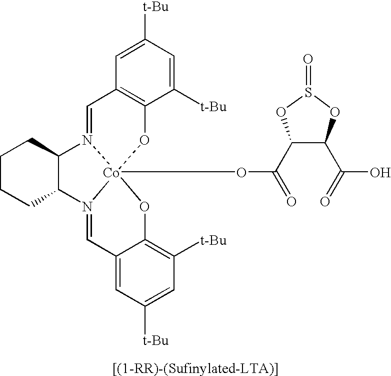

The same method as Preparation Example 5 was conducted except using 9.8 g of 2,3-sulfinyl-L-tartaric acid instead of diacetyl-L-tartaric acid. Then the title compound was obtained quantitatively.

mp 140˜145° C. (dec.)

IR (KBr) 3462, 2955, 2865, 1629, 1610, 1527, 1439, 1362, 1273, 1252, 1204, 1174, 1007, 747 cm⁻¹

### Preparation Example 9

### Preparation of Catalyst [(1-RR)-(Sulfinylated-DTA)]

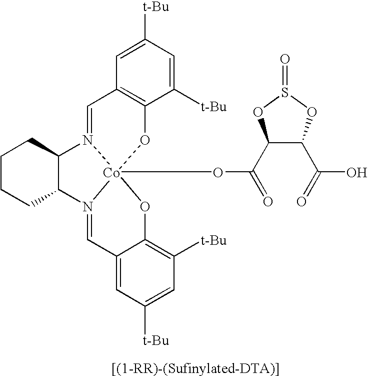

The same method as Preparation Example 5 was conducted except using 9.8 g of 2,3-sulfinyl-D-tartaric acid instead of diacetyl-L-tartaric acid. Then the title compound was obtained quantitatively.

mp 145° C. (dec.)

IR (KBr) 3462, 2955, 2865, 1629, 1610, 1527, 1439, 1362, 1273, 1252, 1204, 1174, 1007, 747 cm⁻¹

### Preparation Example 10

### Preparation of Catalyst [(1-RR)-(Ditosyl-LTA)]

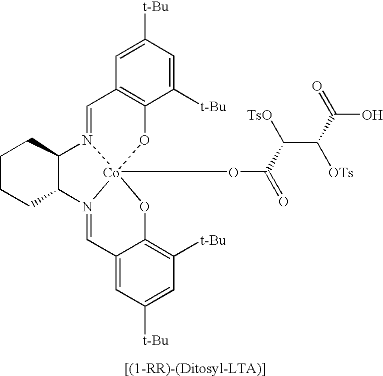

27.8 g of (R,R)—N,N′-bis(3,5-di-t-butylsalicylidene)-1,2-cyclohexanediamine and 16.4 g of cobalt(II)acetate-4H₂O were mixed in 500 ml of ethanol and stirred under reflux for 5 hours. They were filtered at room temperature and cleaned by small amount of ethanol. The obtained solid, 22.9 g of di(p-toluenesulfonyl)-L-tartaric acid, 175 ml of acetone, and 600 ml of dichloromethane were all mixed and stirred for 5 hours with injection of air at room temperature. After the solvent was eliminated under reduced pressure, the title compound was obtained quantitatively.

mp 126° C. (dec.)

IR (KBr) 3429, 2955, 2867, 1714, 1641, 1637, 1531, 1438, 1364, 1355, 1255, 1191, 1177, 1084, 914, 831, 754 cm⁻¹

### Preparation Example 11

### Preparation of Catalyst [(1-RR)-(Dimethyl-LTA)]

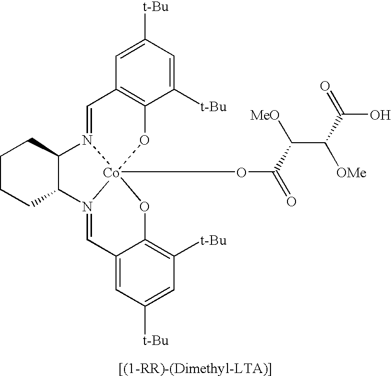

27.8 g of (R,R)—N,N′-bis(3,5-di-t-butylsalicylidene)-1,2-cyclohexanediamine and 16.4 g of cobalt(II)acetate-4H₂O were mixed in 500 ml of ethanol and stirred under reflux for 5 hours. They were filtered at room temperature and cleaned by small amount of ethanol. The obtained solid, 8.9 g of dimethyl-L-tartaric acid, 350 ml of acetone, and 600 ml of dichloromethane were all mixed and stirred for 5 hours with injection of air at room temperature. After the solvent was eliminated under reduced pressure, the title compound was obtained quantitatively.

mp 136° C. (dec.)

IR (KBr) 3444, 2953, 2866, 1743, 1636, 1629, 1529, 1438, 1361, 1255, 1201, 1171, 1106, 1012, 783 cm⁻¹

### Example 1

### Preparation of (S)-epichlorohydrin

46.3 g of racemic epichlorohydrin was mixed with 0.4 mole % of [(1-RR)-(Dibenzoyl-LTA)], which was prepared in Preparation Example 1, and cooled down to 5° C. Then 5.4 g of water was slowly added here and stirred at 20° C. for 3 hours. After fractional distillation under reduced pressure, (S)-epichlorohydrin was obtained with 99.9% ee optical purity and 80% of yield.

### Example 2

### Preparation of (R)-epichlorohydrin

46.3 g of racemic epichlorohydrin was mixed with 0.4 mole % of [(1-SS)-(Dibenzoyl-DTA)], which was prepared in Preparation Example 2, and cooled down to 5° C. Then 5.4 g of water was slowly added here and stirred at 20° C. for 3 hours. After fractional distillation under reduced pressure, (R)-epichlorohydrin was obtained with 99.9% ee optical purity and 80% of yield.

### Example 3-4

### Preparation of (S)-epichlorohydrin using catalyst [(1-RR)-(LTA)] (Preparation Example 7) and [(1-RR)-(Sulfinylated-LTA)] (Preparation Example 8)

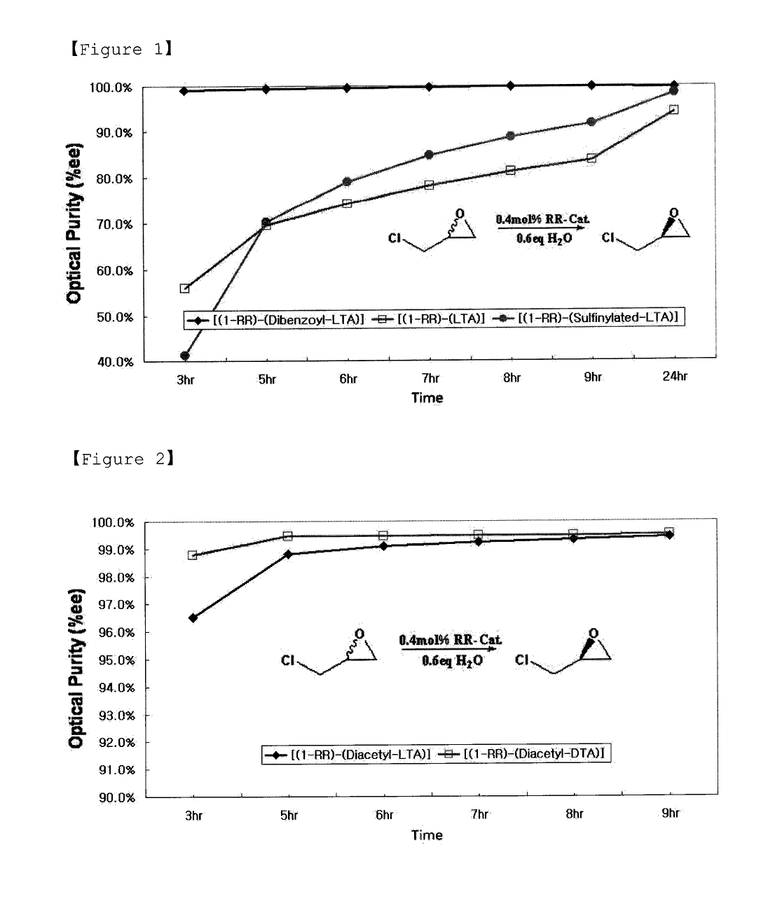

The same method as Example 1 was conducted except using the catalysts prepared in Preparation Example 7 and 8 respectively. The graph showing the change in optical purity depending on reaction time along with that of Example 1 was shown in FIG. 1.

FIG. 1 is a graph showing the difference in reaction rate depending on the location of carboxylic acid group of anionic ligand among the chiral salen catalysts of the present invention. According to FIG. 1, it was found that catalyst [(1-RR)-(Dibenzoyl-LTA)] (Preparation Example 1) had remarkably better reaction rate and stereoselectivity than [(1-RR)-(LTA)] (Preparation Example 7) or [(1-RR)-(Sulfinylated-LTA)] (Preparation Example 8). As shown below, this is because the carboxylic acid groups of [(1-RR)-(Dibenzoyl-LTA)] (Preparation Example 1) are adjacent with each other due to steric hindrance of benzoyloxy groups, but the carboxylic acid groups of [(1-RR)-(LTA)] (Preparation Example 7) or [(1-RR)-(Sulfinylated-LTA)] (Preparation Example 8) are in the opposite direction.

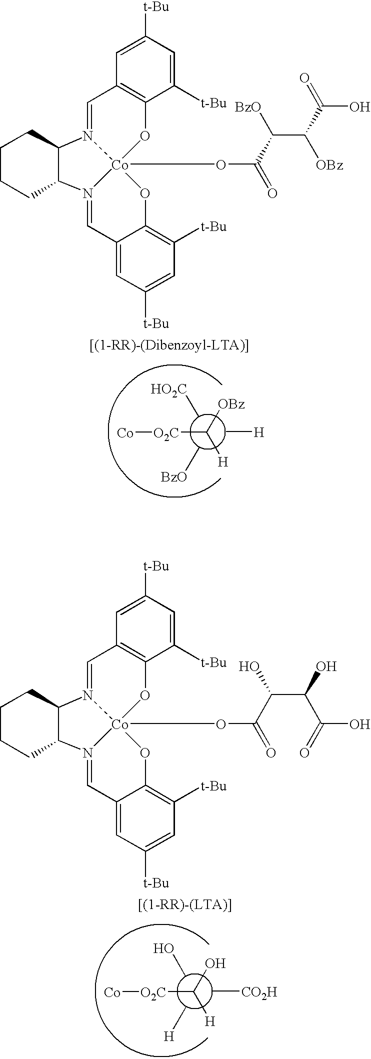

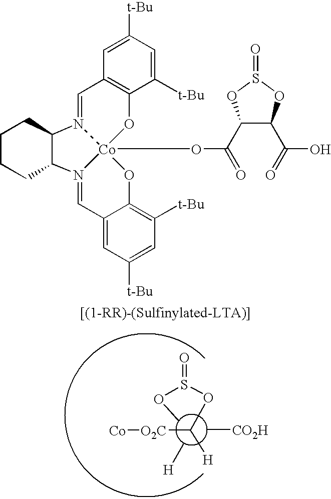

Thus, [(1-RR)-(Dibenzoyl-LTA)] (Preparation Example 1) among the chiral salen catalysts of the present invention gets near the carboxylic acid group when racemic epoxides come near, which promotes ring-opening reaction of racemic epoxides and also improves stereoselectivity.

### Example 5-6

### Preparation of (S)-epichlorohydrin Using Catalyst [(1-RR)-(Diacetyl-LTA)] (Preparation Example 5) and [(1-RR)-(Diacetyl-DTA)] (Preparation Example 6)

The same method as Example 1 was conducted except using the catalysts prepared in Preparation Example 5 and 6. The graph showing the change in optical purity by reaction time was shown in FIG. 2.

FIG. 2 is the graph showing the difference in reaction rate depending on each catalyst's structure to see the change depending on the stereostructure of anionic ligand among the chiral salen catalysts of the present invention. According to FIG. 2, it was found that [(1-RR)-(Diacetyl-LTA)] (Preparation Example 5) and [(1-RR)-(Diacetyl-DTA)] (Preparation Example 6) had little difference in reaction rate and stereoselectivity. As shown below, the carboxylic acid groups of [(1-RR)-(Diacetyl-LTA)] (Preparation Example 5) and [(1-RR)-(Diacetyl-DTA)] (Preparation Example 6) are all adjacent with each other due to steric hindrance of acetyloxy groups, so the racemic epoxides approach near carboxylic acid groups of both catalysts. Therefore, there is almost no difference in the degree of ring-opening of recemic epoxides.

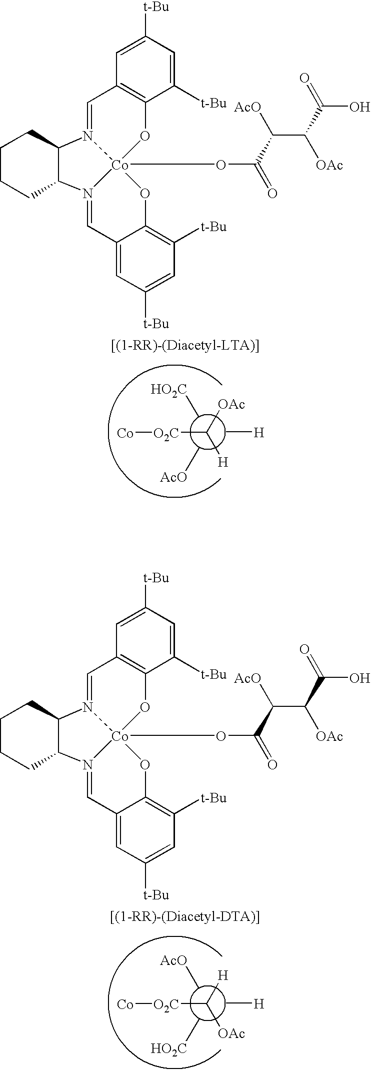

### Example 7-10

### Preparation of (S)-epichlorohydrin Using Catalysts [(1-RR)-(Succinic acid)], [(1-RR)-(Glutaric acid)], [(1-RR)-(Adipic acid)], and [(1-RR)-(Pimeric acid)]

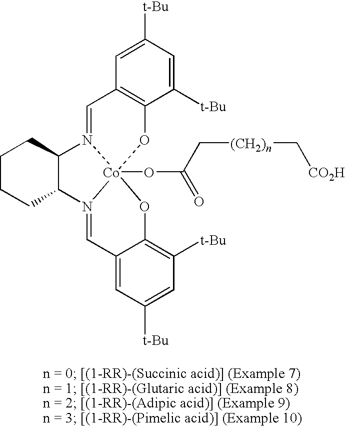

1.8 g of (R,R)—N,N′-bis(3,5-di-t-butylsalicylidene)-1,2-cyclohexanediamine and 1.1 g of cobalt(II)acetate-4H₂O were mixed in 35 ml of ethanol and stirred under reflux for 5 hours. They were filtered at room temperature and cleaned by small amount of ethanol. The obtained solid was uniformly divided into four parts and mixed with 6 ml of acetone and 10 ml of dichloromethane respectively. Also, 1.0 equivalent weight of succinic acid, glutaric acid, adipic acid, and pimeric acid were added respectively and stirred for 3 hours with injection of air at room temperature. After the solvent was eliminated under reduced pressure, the title compounds were obtained quantitatively.

Each catalyst was mixed into 15.3 g of racemic epichlorohydrin and cooled down to 5° C. With 1.6 g of water slowly being added, reaction was done at 20° C. The graph showing the change in optical purity by reaction time was shown in FIG. 3.

FIG. 3 is the graph showing the difference in reaction rate depending on each catalyst's structure to see the change depending on the length of anionic ligand among the chiral salen catalysts of the present invention. According to FIG. 3, it was found that there was little difference in reaction rate depending on the length of anionic ligand. When there was no steric hindrance of anionic ligand, the acid in carboxylic acid groups did not play a big role, so it was found that the suitable three dimensional structure of anionic ligand affects the overall activity or stereoselectivity of catalysts.

### Comparative Example 1

### Comparing the Change in Optical Purity of (S)-epichlorohydrin

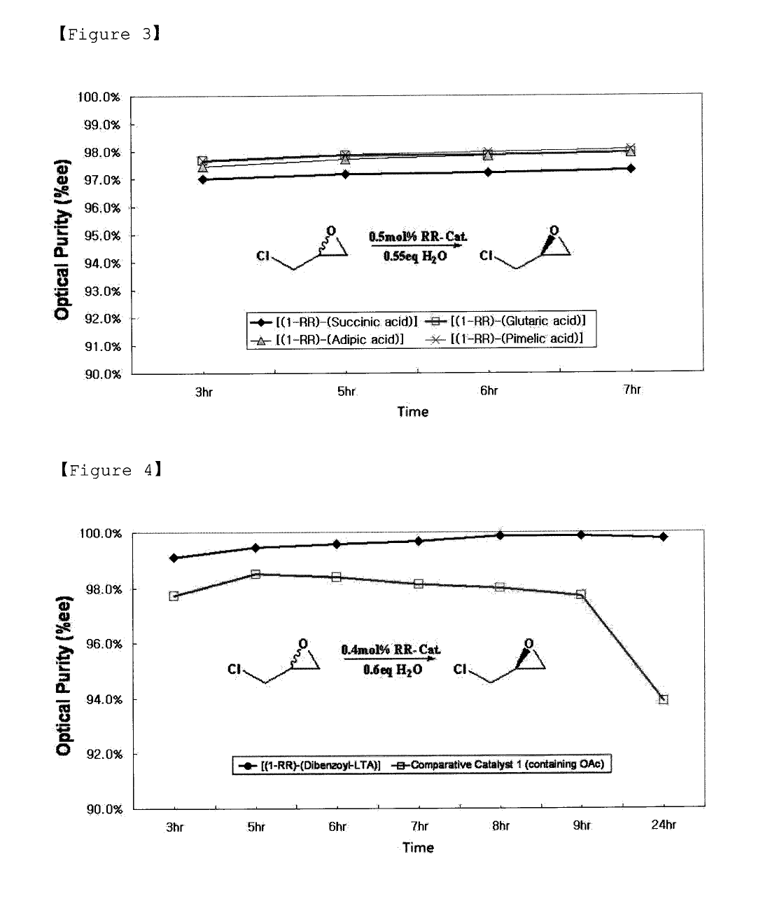

46.3 g of each racemic epichlorohydrin was mixed with 0.4 mole % of the catalyst [(1-RR)-(Dibenzoyl-LTA)] prepared in Preparation Example 1 and chiral salen catalyst (Comparative Catalyst 1) containing the existing acetic acid groups, and cooled down to 5° C. With 5.4 g of water slowly being added, reaction was done at 20° C. The graph showing the change in optical purity by reaction time was shown in FIG. 4.

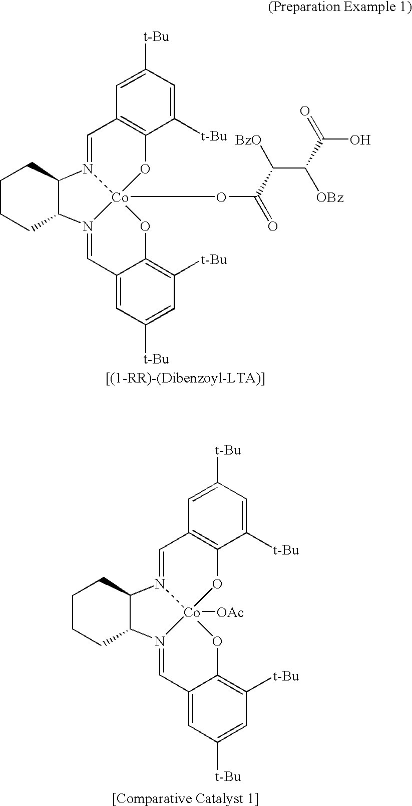

FIG. 4 is the graph showing how much the representative chiral salen catalyst [(1-RR)-(Dibenzoyl-LTA)] (Preparation Example 1) of the present invention and the existing representative chiral salen catalyst (Comparative Catalyst 1) containing acetic acid group (OAc) by Eric N. Jacobsen affect racemization of chiral epoxides generated after reaction. According to FIG. 4, when the chiral salen catalyst [(1-RR)-(Dibenzoyl-LTA)] (Preparation Example 1) of the present invention was used, little racemization was occurred as time passed, but the existing representative chiral salen catalyst (Comparative Catalyst 1) containing acetic acid group (OAc) by Eric N. Jacobsen, which is relatively unstable at the temperature, was used, the optical purity deteriorated as time passed. Thus, it takes a lot of time to purify desired materials after reaction when mass manufacturing. When the new chiral salen catalyst by the present invention is used, chiral epoxides having high optical purity can be obtained even after purification, but when the existing chiral salen catalysts (Comparative Catalyst 1) containing acetic acid group is used, racemization occurs while purifying the reactant or getting ready for purification, which inhibits obtaining chiral epoxides with high optical purity.

### Example 11

### Repetitive Preparation of (S)-epichlorohydrin

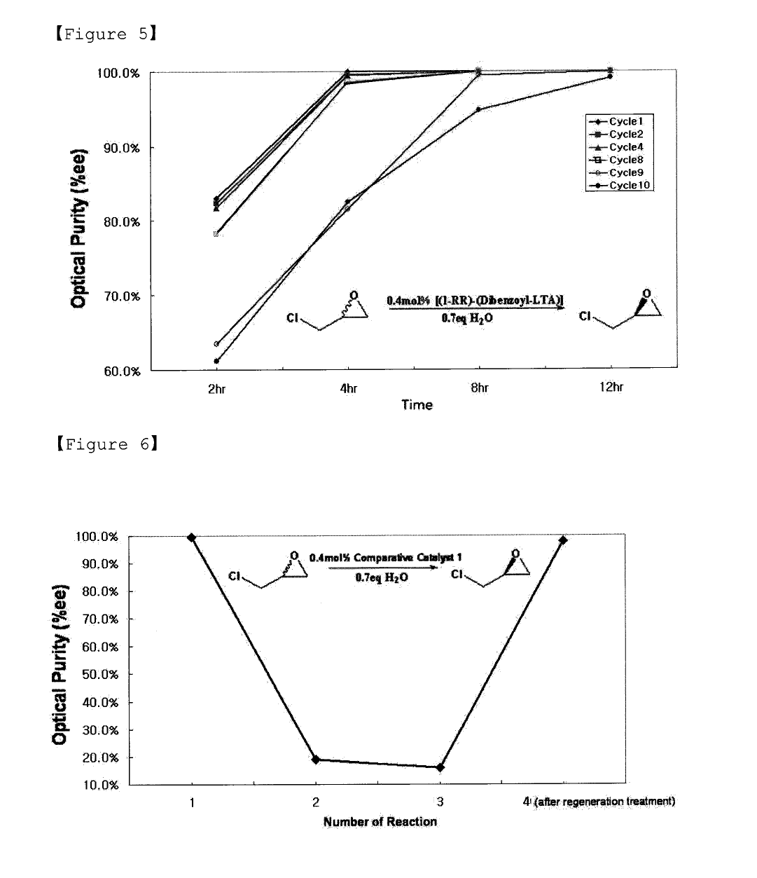

46.3 g of racemic epichlorohydrin was mixed with 0.4 mole % of [(1-RR)-(Dibenzoyl-LTA)], which was prepared in Preparation Example 1, and cooled down to 5° C. Then 6.3 g of water was slowly added here and stirred at 20° C. for 4 hours. After fractional distillation under reduced pressure, (S)-epichlorohydrin was obtained with 99.9% ee optical purity. Then, after new racemic epichlorohydrin and water were added, the same reaction was repeated ten times to obtain (S)-epichlorohydrin with more than 99.1% ee optical purity. The graph showing the change in optical purity by reaction time was shown in FIG. 5.

### Comparative Example 2

### Repetitive Preparation of (S)-epichlorohydrin

Using 0.4 mole % of the existing chiral salen catalyst (Comparative Catalyst 1) containing acetic acid groups, (S)-epichlorohydrin was obtained through the same reaction as Example 11. Then, the used catalyst was reacted once again with no acetic acid treatment to obtain (S)-epichlorohydrin of 19% ee. One more reaction of the used catalyst was followed with no acetic acid treatment to obtain (S)-epichlorohydrin of 16% ee. After the third reaction, by the disclosed method to regenerate the catalyst [Science, Vol. 277, 936, 1997], toluene and 2 mole ratio of acetic acid were added and stirred at room temperature for 5 hours with the air injected. After the solvent was eliminated under reduced pressure, the regenerated catalyst was obtained. When the reactions were performed on the same condition using this catalyst, the duration time for reaction increased from 4 hours to 8 hours at the first reaction and the optical purity of (S)-epichlorohydrin decreased to 98% ee. The graph showing the change in optical purity by reaction time was shown in FIG. 6.

FIG. 5 is the graph showing the change of stereoselectivity depending on reaction rate and reuse number of the representative chiral salen catalyst [(1-RR)-(Dibenzoyl-LTA)] (Preparation Example 1) of the present invention, and FIG. 6 is the graph showing the change of stereoselectivity depending on reuse of the existing chiral salen catalyst (Comparative Catalyst 1) containing acetic acid groups (OAc).

According to FIGS. 5 and 6, when the chiral salen catalyst [(1-RR)-(Dibenzoyl-LTA)] (Preparation Example 1) of the present invention was used, it is possible to obtain the chiral epoxides with higher isomer selectivity (99% ee or more) than that of Comparative Catalyst 1, and it was also found that it did not lose catalytic activity and could be reused repetitively with no regeneration treatment. Comparative Catalyst 1, on the other hand, lost its activity after it was used once, and showed enough activity only after additional catalyst regeneration processes (e.g. oxidation process by acetic acid treatment). Even after the regeneration treatment by acetic acid, it was hard to get chiral epoxides with more than 99% ee and the reaction took much longer time than the case using the new catalyst (Comparative Example 2).

### Comparative Example 3

### Comparing the Change in Optical Purity of (S)-epichlorohydrin Using Catalyst [(1-RR)-(Diacetyl-LTA)] (Preparation Example 5) and Comparative Catalyst 2

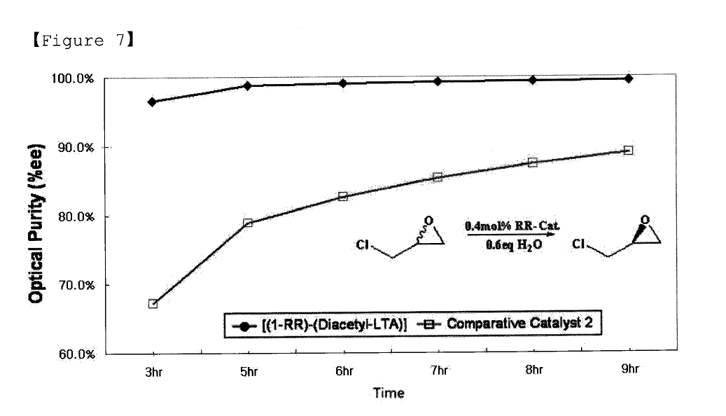

46.3 g of each racemic epichlorohydrin was mixed with 0.4 mole % of the catalyst [(1-RR)-(Diacetyl-LTA)] prepared in Preparation Example 5 and the catalyst (Comparative Catalyst 2) that carboxylic acid group of the said catalyst is neutralized, and cooled down to 5° C. Then 5.4 g of water was slowly added here and reaction was done at 20° C. The graph showing the change in optical purity by reaction time was shown in FIG. 7.

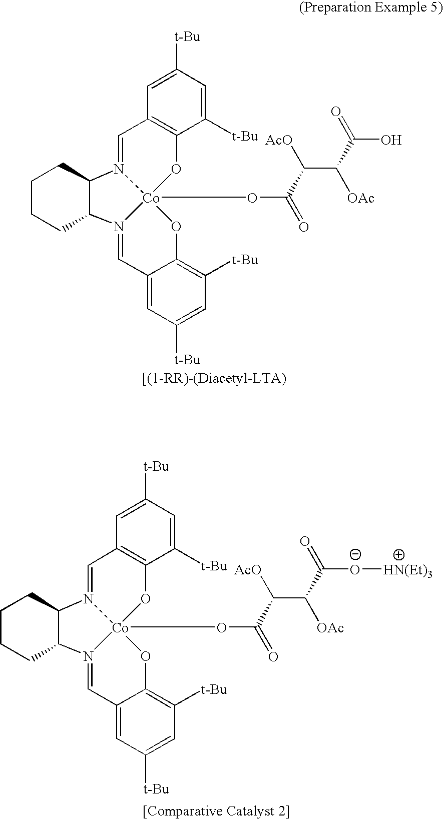

FIG. 7 is the graph showing the difference of reaction rate depending on each catalyst structure to see the role of acid in carboxylic acid group of anionic ligand among the chiral salen catalysts of the present invention.

According to FIG. 7, compared to the representative chiral salen catalyst of the present invention [(1-RR)-(Diacetyl-LTA)] (Preparation Example 5), Comparative Catalyst 2 having no carboxylic acid groups in anionic ligand showed remarkably lowered reaction rate. Thus, it is found that the acid in carboxylic acid groups affects the reaction rate and also involves in stereoselectivity.

### Example 12-28

### Preparation of (R)-1,2-epoxy (or (S)-1,2-epoxy) Compounds

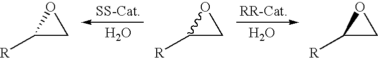

0.5 mole of racemic 1,2-epoxy compounds were added with 0.4 mole % of the catalyst prepared in Preparation Example 1 [(1-RR)-(Dibenzoyl-LTA)] (or Preparation Example 2 [(1-SS)-(Dibenzoyl-DTA)]) and cooled down to 5° C. Then 5.4 g of water was slowly added here and stirred at 20° C. After fractional distillation under reduced pressure, the target compounds were obtained with more than 99% ee optical purity

TABLE 1

| Example | R | Optical purity |
|---|---|---|
| 12 | —CH₃ | >99% ee |
| 13 | —CH₂CH₃ | >99% ee |
| 14 | —CH₂CH₂CH₂CH₃ | >99% ee |
| 15 | —C(CH₃)₃ | >99% ee |
| 16 | Benzyl | >99% ee |
| 17 | cyclohexyl | >99% ee |
| 18 | 3-butenyl | >99% ee |
| 19 | —CH₂F | >99% ee |
| 20 | —CF₃ | >99% ee |
| 21 | —CH₂OMe | >99% ee |
| 22 | —CH₂OPh | >99% ee |
| 23 | —CH₂OBn | >99% ee |
| 24 | —CH₂OCO(CH₂)₃ | >99% ee |
| 25 | —CH₂CO₂Et | >99% ee |
| 26 | —CH₂NHBoc | >99% ee |
| 27 | —COCH₃ | >99% ee |
| 28 | —CO₂Me | >99% ee |

### Example 29-31

### Preparation of (R)-aryl-1,2-epoxy (or (S)-aryl-1,2-epoxy) Compounds

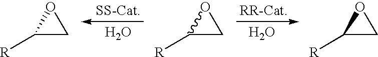

0.5 mole of racemic aryl-1,2-epoxy compounds were added with 0.6 mole % of the catalyst prepared in Preparation Example 1 [(1-RR)-(Dibenzoyl-LTA)] (or Preparation Example 2 [(1-SS)-(Dibenzoyl-DTA)]) and cooled down to 5° C. Then 5.4 g of water was slowly added here and stirred at 20° C. After fractional distillation under reduced pressure, the target compounds were obtained with more than 99% ee optical purity

TABLE 2

| Example | R | Optical purity |
|---|---|---|
| 29 | phenyl | >99% ee |
| 30 | 3-chlorophenyl | >99% ee |
| 31 | 4-fluorophenyl | >99% ee |

### Example 32

### Preparation of (R)-1,2-butanediol (or (S)-1,2-butanediol)

72 g of racemic 1,2-epoxybutane was added with 0.4 mole % of the catalyst prepared in Preparation Example 1 [(1-RR)-(Dibenzoyl-LTA)] (or Preparation Example 2 [(1-SS)-(Dibenzoyl-DTA)]) and cooled down to 5° C. Then 5.4 g of water was slowly added here and stirred at 20° C. for 2.5 hours. The remaining 1,2-epoxybutane was eliminated under reduced pressure and the remains were added with dichloromethane and water. After separation of the water layer and fractional distillation, (R)-1,2-butanediol (or (S)-1,2-butanediol) were obtained with 98.5% ee optical purity and 55% of yield.

### Example 33

### Mass Manufacturing of (S)-epichlorohydrin

463 kg of racemic epichlorohydrin was added with 19.2 kg of the catalyst prepared in Preparation Example 1 [(1-RR)-(Dibenzoyl-LTA)] and cooled down to 5° C. Then 49.5 kg of water was slowly added here and stirred at 20° C. for 7 hours. The reactant was thin film evaporated at the temperature lower than 30° C. under reduced pressure to obtain 190 kg of (S)-epichlorohydrin (99.8% ee optical purity, 82% of yield). The remains were added with new racemic epichlorohydrin and water and the same reaction was repeated to obtain (S)-epichlorohydrin with more than 99.0% ee optical purity and 80% of average yield.

### INDUSTRIAL APPLICABILITY

The chiral salen catalysts of the present invention are newly structured catalysts having carboxylic acid groups, which are different from the existing chiral salen catalysts, and they can be reused by overcoming the disadvantages of the existing chiral salen catalysts. Also, they are useful catalysts for stereoselective hydrolysis reaction that can mass manufactures stereoselective chiral epoxides or chiral 1,2-diol from racemic epoxides with high optical purity and high yield.

1. New chiral salen catalysts presented as Chemical Formula 1 as below. 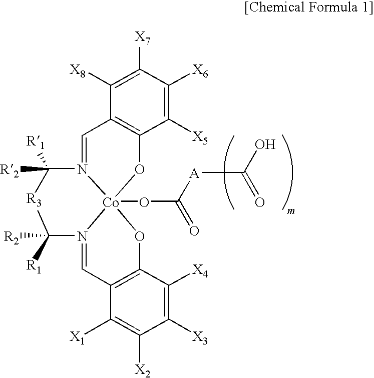
In Chemical Formula 1:
R₁, R₂, R′₁, R′₂, X₁, X₂, X₃, X₄, X₅, X₆, X₇ and X₈, are independently hydrogen atom, linear or branched saturated or unsaturated (C1-C7) alkyl group, (C1-C7) alkoxy group, halogen atom, hydroxy group, amino group, thiol group, nitro group, aminocarbonyl, (C3-C7) cycloalkyl, (C1-C7)alkoxy(C1-C7)alkyl, (C1-C7) alkylcarbonyl, (C1-C7) alkoxycarbonyl, (C3-C7)cycloalkyl(C1-C7)alkoxy, mono or di (C1-C7)alkylamino, (C1-C7) alkylcarbonylamino, t-butoxycarbonylamino, phthalimido, carboxylic group, aldehyde group, (C1-C7) alkylthio, (C1-C7) alkylsulfonyl group, tri(C1-C7) alkylsilyl group, tri(C6-C10)arylsilyl group, mono or di (C1-C7) alkylaminocarbonyl, —(CH₂)ₖ—R₄, or (C2-C10)alkylene to form a ring by combining with adjacent substituents;
R₃ is a direct bond, (C1-C5)alkylene, —NH—, —O—, or —S—;
R₄ is a 3 to 5-membered saturated or unsaturated heterocycle including N, O, or S, (C3-C12) cycloalkyl, or phenyl;
A is (C1-C12)alkylene, which can be more substituted with linear or branched saturated or unsaturated (C1-C7) alkyl group, (C1-C7) alkoxy group, halogen atom, hydroxy group, amino group, thiol group, (C1-C7) alkylcarbonylamino, t-butoxycarbonylamino, phthalimido, —O₂CY₃, or —O₃SY₃, or can form cycles by being connected with (C2-C10)alkylene, —OSO₂—, —OSO₃—, or —OCO₂—;
Y₃ is a linear or branched saturated or unsaturated (C1-C7)alkyl group or phenyl, which can be more substituted with linear or branched saturated or unsaturated (C1-C7)alkyl group, halogen, or nitro;
k is an integer of 0 to 8;
m is an integer of 1 to 3.]

2. The new chiral salen catalysts of claim 1, wherein said chiral salen catalysts are presented as Chemical Formula 2 as below. 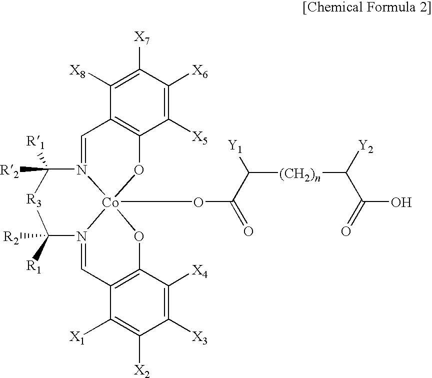
[In Chemical Formula 2:
R₁, R₂, R₃, R′₁, R′₂, X₁, X₂, X₃, X₄, X₅, X₆, X₇ and X₈ are identical as those of said claim 1;
Y₁ and Y₂ are independently hydrogen atom, linear or branched saturated or unsaturated (C1-C7) alkyl group, (C1-C7)alkoxy group, halogen atom, hydroxy group, amino group, thiol group, (C1-C7)alkylcarbonylamino, t-butoxycarbonylamino, phthalimido, —O₂CY₃, or —O₃SY₃, or Y₁ and Y₂ can form cycles by being connected with (C2-C10)alkylene, —OSO₂—, —OSO₃—, or —OCO₂—;
Y₃ is a linear or branched saturated or unsaturated (C1-C7) alkyl group or phenyl, which can be substituted more with linear or branched saturated or unsaturated (C1-C7) alkyl group, halogen, or nitro;
n is an integer of 0 to 10.]

3. The new chiral salen catalyst of claim 1, wherein said chiral salen catalysts are the catalysts for reaction to prepare chiral compounds such as chiral epoxides or chiral 1,2-diol from racemic epoxides.

4. The new chiral salen catalysts of claim 2, wherein said X₁, X₂, X₃, X₄, X₅, X₆, X₇ and X₃ are independently selected from the group consisting of hydrogen atom, linear or branched saturated or unsaturated (C1-C7) alkyl group and (C1-C7) alkoxy group.

5. The new chiral salen catalysts of claim 4, wherein said X₁, X₂, X₃, X₄, X₅, X₅, X₇ and X₈ are independently hydrogen atom or t-butyl group.

6. The new chiral salen catalysts of claim 2, wherein R₁ and are combined to (C2-C8)alkylene to form a ring and R₂ and R′₂ are hydrogen atoms; or R₂ and R′₂ are combined to (C2-C8)alkylene to form a ring and R₁ and R′₁ are hydrogen atoms.

7. The new chiral salen catalysts of claim 2, wherein Y₁ and Y₂ are independently hydrogen atom, (C1-C7)alkoxy group, halogen atom, hydroxy group, —O₂CY₃ or —O₃SY₃, or Y₁ and Y₂ can form cycles by being connected with —OSO₂—, —OSO₃— or —OCO₂—; Y₃ is a linear or branched saturated or unsaturated (C1-C7) alkyl group, phenyl or nitrophenyl, in which said alkyl group or phenyl can be substituted more with linear or branched saturated or unsaturated (C1-C7) alkyl group or halogen.

8. The new chiral salen catalysts of claim 7, wherein Y₁ and Y₂ are independently (C1-C7)alkoxy group, —O₂CY₃ or —O₃SY₃, and Y₃ is a linear or branched saturated or unsaturated (C1-C7) alkyl group or phenyl, in which said alkyl group or phenyl can be substituted more with linear or branched saturated or unsaturated (C1-C7) alkyl group or halogen.

9. Preparation methods of chiral salen catalysts presented as Chemical Formula 1 of claim 1 prepared by reacting the compounds presented as Chemical Formula 3 and the compounds presented as Chemical Formula 4. 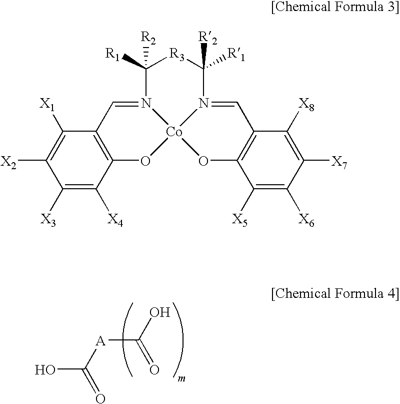
[In Chemical Formula 3 and 4: R₁, R₂, R₃, R′₁, R′₂, X₁, X₂, X₃, X₄, X₅, X₈, X₇, X₈, A and m are identical as those of said claim 1.]

10. The preparation methods of chiral salen catalysts of claim 9, wherein said X₁, X₂, X₃, X₄, X₅, X₆, X₇ and X₈ are independently selected from the group consisting of hydrogen atom, linear or branched saturated or unsaturated (C1-C7) alkyl group and (C1-C7) alkoxy group.

11. The preparation methods of chiral salen catalysts of claim 10, wherein said X₁, X₂, X₃, X₄, X₅, X₆, X₇ and X₈ are independently hydrogen atom or t-butyl group.

12. The preparation methods of chiral salen catalysts of claim 9, wherein R₁ and R′₁ are combined to (C2-C8)alkylene to form a ring and R₂ and R′₂ are hydrogen atoms; or R₂ and R′₂ are combined to (C2-C8)alkylene to form a ring and R₁ and R′₁ are hydrogen atoms.

13. Preparation methods of chiral compounds such as chiral epoxides or chiral 1,2-diol by stereoselective hydrolysis of racemic epoxides using the chiral salen catalysts presented as Chemical Formula 1 as reaction catalysts. 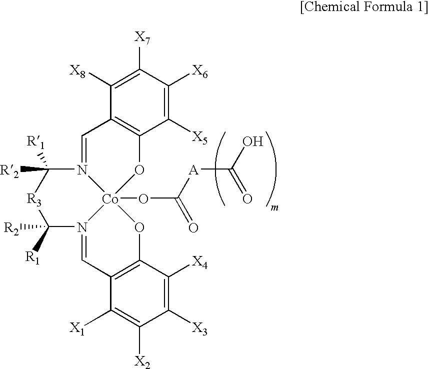
[Said R₁, R₂, R₃, R′₁, R′₂, X₁, X₂, X₃, X₄, X₅, X₆, X₇, X₈, A and m are identical as those of said claim 1.]

14. The preparation methods of chiral compounds of claim 13, wherein the chiral salen catalysts presented as Chemical Formula 2 are used as reaction catalysts. 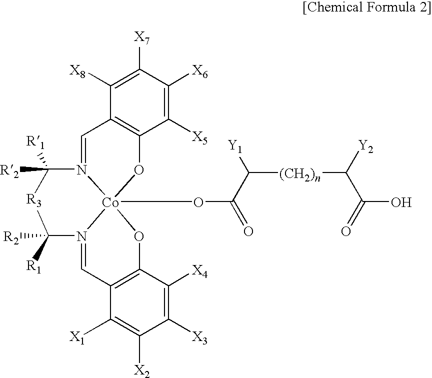
[Said R₁, R₂, R₃, R′₁, R′₂, X₁, X₂, X₃, X₄, X₅, X₆, X₇ and X₈ are identical as those of said claim 1; Y₁, Y₂ and n are identical as those of said claim 2.]

15. The preparation methods of chiral compounds of claim 14, wherein said X₁, X₂, X₃, X₄, X₅, X₆, X₇ and X₈ are independently selected from the group consisting of hydrogen atom, linear or branched saturated or unsaturated (C1-C7) alkyl group and (C1-C7) alkoxy group.

16. The preparation methods of chiral compounds of claim 14, wherein said X₁, X₂, X₃, X₄, X₅, X₆, X₇ and X₈ are independently hydrogen atom or t-butyl group.

17. The preparation methods of chiral compounds of claim 14, wherein R₁ and R′₁ are combined to (C2-C8)alkylene to form a ring and R₂ and R′₂ are hydrogen atoms; or R₂ and R′₂ are combined to (C2-C8)alkylene to form a ring and R₁ and R′₁ are hydrogen atoms.

18. The preparation methods of chiral compounds of claim 14, wherein Y₁ and Y₂ are independently hydrogen atom, (C1-C7)alkoxy group, halogen atom, hydroxy group, —O₂CY₃ or —O₃SY₃, or Y₁ and Y₂ can form cycles by being connected with —OSO₂—, —OSO₃— or —OCO₂—; Y₃ is a linear or branched saturated or unsaturated (C1-C7) alkyl group, phenyl or nitrophenyl, in which said alkyl group or phenyl can be substituted more with linear or branched saturated or unsaturated (C1-C7) alkyl group or halogen.

19. The preparation methods of chiral compounds of claim 18, wherein Y₁ and Y₂ are independently (C1-C7)alkoxy group, —O₂CY₃ or —O₃SY₃, and Y₃ is a linear or branched saturated or unsaturated (C1-C7) alkyl group or phenyl, in which said alkyl group or phenyl can be substituted more with linear or branched saturated or unsaturated (C1-C7) alkyl group or halogen.

20. The preparation methods of chiral compounds of claim 13, wherein said racemic epoxides are presented as Chemical Formula 5. 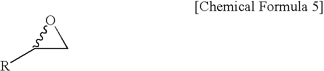
[In Chemical Formula 5:
R is a linear or branched saturated or unsaturated (C1-C7) alkyl group, (C3-C7)cycloalkyl group, (C1-C7)alkoxy group, phenyl group, carboxylic group, aldehyde group, (C3-C7)cycloalkyl, (C1-C7)alkoxy(C1-C7)alkyl, (C1-C7)alkylcarbonyl, (C1-C7)alkoxycarbonyl, (C3-C7)cycloalkyl(C1-C7)alkoxy, (C1-C7)alkylsulfonyl group, or —(CH₂)ₖ—R₅; the said alkyl, cycloalkyl, alkoxy, or phenyl can be substituted more with halogen;
R₅ is a linear or branched saturated or unsaturated (C1-C7) alkyl group, (C1-C7)alkoxy group, phenyl group, (C3-C7)cycloalkyl group, 3 to 5-membered saturated or unsaturated heterocycle including N, O, or S, halogen atom, hydroxy group, amino group, thiol group, nitro group, aminocarbonyl, mono or di(C1-C7)alkylaminocarbonyl, (C3-C7)cycloalkyl, (C1-C7)alkoxy(C1-C7)alkyl, (C1-C7)alkylcarbonyl, (C1-C7)alkoxycarbonyl, (C3-C7)cycloalkyl(C1-C7)alkoxy, (C6-C10)aryloxy, benzyloxy, (C1-C7)alkylcarbonyloxy, mono or di(C1-C7)alkylamino, (C1-C7)alkylcarbonylamino, t-butoxycarbonylamino, phthalimido, carboxylic group, aldehyde group, (C1-C7)alkylthio, (C1-C7)alkylsulfonyl group, tri(C1-C7)alkylsilyl group, or tri(C6-C10)arylsilyl group;
k is an integer of 0 to 8.]

21. (canceled)

22. (canceled)
